(19) United States
(12) Patent Application Publication
(10) Pub. No.: US 2004/0142347 A1
(43) Pub. Date: Jul. 22, 2004

(54) Mitochondrial DNA autoscoring system

(76) Inventors: Timothy B. Stockwell, Chantilly, VA (US); Stephen A. Glanowski, Great Falls, VA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/672,937

(22) Filed: Sep. 26, 2003

Related U.S. Application Data
(60) Provisional application No. 60/414,815, filed on Sep. 26, 2002.

(51) Int. Cl. C12Q 1/68; G06F 19/00; G01N 33/48; G01N 33/50

(52) U.S. Cl. 435/6; 702/20

(57) ABSTRACT

An analysis approach used to evaluate base-call ambiguity and quality for a selected sequence. The methods described herein may be adapted to automated procedures for sequence acquisition, evaluation, and sample identification. In particular, the disclosed methods may be adapted to sequence identification techniques such as forensic analysis to provide an automated approach to data interpretation thereby reducing or eliminating the need for detailed investigator review. Implementation of these automated methods desirably provides a means by which to improve the speed and accuracy of the analysis as compared to conventional methods and may be used to improve identification throughput especially in large or complex sequencing projects.

### PRIORITY

This application claims priority under 35 U.S.C. § 119 to U.S. Provisional Application No. 60/414815, filed on Sep. 26, 2002, entitled METHOD TO DETERMINE AMBIGUOUS BASES IN GENETIC SEQUENCES.

### BACKGROUND

1. Field

The present teachings generally relate to a system and methods for large-scale sequencing, and more particularly to an automated data analysis system for providing reliable mitochondrial DNA (mtDNA) profiles for use in data analysis and quality assessment.

2. Description of the Related Art

The use of mitochondrial DNA (mtDNA) for human identification in a forensic setting has now become an important adjunct to more commonly used nuclear DNA analysis techniques. Identification through mtDNA analysis has certain advantages in that it is present in relatively high concentrations (e.g. hundreds or thousands of copies per cell) and tends to withstand environmental challenges that may cause nuclear DNA damage, thus providing improved sensitivity in compromised or degraded samples.

Despite these desirable characteristics a principle difficulty in conventional mtDNA sequencing and analysis is that it is labor intensive, requiring careful evaluation by forensic examiners to determine the mtDNA profile. In instances where the number of samples to be analyzed is large, conventional methods for mtDNA analysis may prove to be inadequate especially in terms of turnaround time and throughput. Consequently, there is a need for improved methods for analyzing mtDNA especially in the context of large scale sample processing. Furthermore, there is a need for methods of mtDNA analysis that are semi-or-fully automated to reduce the dependence on detailed investigator review and interpretation of sample sequencing data.

### SUMMARY

In various embodiments, the present teachings described herein disclose methods for high-throughput processing of DNA sequence data and information. The DNA sequence information can be, for example, mtDNA sequence data or nuclear DNA sequence data. In certain embodiments, the system and methods further provide a high-throughput platform for evaluating mtDNA sequence data from large-scale forensic sequencing programs. The sequence data can be obtained from automated sequencers, or base calling systems, and other technologies and methodologies known in the art. In various embodiments, the sequence analysis approach of the present teachings can be adapted for use with conventional sequence analysis software including TraceTuner, phred, phrap, blast, fasta, and others.

In other embodiments, the present teachings relate to an automated data analysis system and methods to provide reliable mtDNA profiles useful for assessing sequence data quality. In still other embodiments, the automated system and methods provide reliable mtDNA profiles useful for comparison of victim samples with reference samples. Further, certain embodiments provide an automated system and methods for reporting mtDNA profiles according to accepted standards used by the forensic community, including standardized nomenclature. Still other embodiments provide an automated system and methods for scoring mtDNA samples against known standards and controls.

In other embodiments, the present teachings described herein provide for an automated data analysis approach that efficiently coordinates the activities of mtDNA analysis including the collection of relevant sequence information, analysis of the sequence information, assembly of consensus sequences, filtering of consensus sequences based on sequence quality and coverage, and reporting of analysis results including identification of sequence variants. In certain instances, sequence variants can be identified by comparing to known standards of references and may include, for example, the original Cambridge Reference Sequence (CRS) (Anderson et al., Nature (1981)), or the revised Cambridge Reference Sequence (rCRS) of the mitochondrial genome (Andrews et al., Nat. Genet. (1999)).

In still other embodiments, the system may be adapted for use with automated sequencers or base calling systems, sequence assembly systems, and post-processing systems. Examples of base calling systems and methods known in the art, include, but are not limited to, phred (Ewing, B., et al. Genome Research 8:175-185, 1998)] Examples of sequence assembly systems and methods known in the art, include, but are not limited to, phrap (www.phrap.org). In certain embodiments, the post-processing systems and modules automate data analysis by implementing rules typically embodied in procedures for manual data review. Examples of rules typically embodied in standard procedures for manual review include, but are not limited to, coverage requirements, definitions of background versus mixed base calls, detection of mixtures and heteroplasmic site rules.

Other embodiments of the present teachings provide methods for high-throughput analysis of mtDNA sequence data. These methods may include, for example, coordinating the collection of the raw sequence data, or sequence traces, from sequencing runs; analyzing the raw sequence data, or sequence traces, from sequencing runs; assembling consensus sequences; comparing consensus sequences to known standards of reference sequences; defining reportable ranges within the consensus sequences based on quality and coverage statistics for obtained or generated the sequence data; and reporting sequence information including sequence variants.

In some embodiments, the disclosed methods may further include incorporating automated data analysis rules or procedures derived from manual review techniques associated with analysis mtDNA sequence data. Examples of data analysis rules used in manual review of mtDNA sequence data include, but are not limited to, rules regarding coverage requirements, definitions of background versus mixed base calls, detection of mixtures and heteroplasmic site rules.

In one aspect, the present teachings provide an automated method for sequence evaluation used to compare sequence information relating to at least one sample against sequence information relating to at least one reference. The method further comprises the steps of: (i) acquiring sequence information relating to the at least one sample and to the at least one reference; (ii) evaluating the sequence information relating to the at least one sample to identify ambiguous bases present within the sample sequence information by applying a rule-based criteria wherein ambiguous bases are distinguished from unambiguous bases on the basis of the following criteria: (a) scan position differences, (b) peak height ratios, (c) peak area ratios, and (d) base composition; and (iii) evaluating the quality and coverage of the sample sequence information in comparison to the reference sequence information to identify reportable ranges and sequence variants for the sample sequence information.

In another aspect, the present teachings provide an automated method for mitochondrial DNA analysis used to identify associations between a target sample of unknown familial origin with that of at least one reference sample. The method further comprises the steps of: (i) acquiring genetic information describing the sequence composition and characteristics for a plurality of nucleotides relating to the mitochondrial genetic makeup of the target sample and at least one reference sample; (ii) assessing the genetic information to identify a degree of ambiguity associated with each of the plurality of nucleotides wherein ambiguous nucleotides are distinguished from unambiguous nucleotides on the basis of: (a) scan position differences, (b) peak height ratios, (c) peak area ratios, and (d) nucleotide compositions; (iii) comparing the genetic information and the degree of ambiguity associated with each of the plurality of nucleotides of the target sample and the at least one reference sample to identify a nucleotide signature that provides distinguishing information used to identify sequence similarities and differences between the target sample and the at least one reference sample; and (iv) comparing the nucleotide signature of the target sample to that of the at least one reference sample such that substantially identical nucleotide signatures identify the target sample as being of the same familial origin as the at least one reference sample and nucleotide signatures which are not substantially identical identify the target sample as not being of the same familial origin as the at least one reference sample.

In still other embodiments the present teachings provide a system for conducting automated comparison analyses of sequence information relating to at least one sample and at least one reference, the system comprising: a setup module that acquires and formats sequence information relating to the at least one sample and the at least one reference; a trace-analysis module that prepares the sequence information for comparison and includes functionality to select appropriate regions of the sequence information of the at least one sample and the at least one reference for subsequent comparison; an assembly-analysis module that generates one or more consensus sequences between the at least on sample and the at least one reference and includes functionality for evaluating the sequence information to distinguish between ambiguous and unambiguous nucleotides within the sequence information on the basis of the following criteria: (a) scan position differences, (b) peak height ratios, (c) peak area ratios, and (d) nucleotide composition; and a variant-analysis module that generates a nucleotide profile which details the results of comparing the at least one sample and the at least one reference and identifies nucleotide variations between the at least one sample and the at least one reference wherein the variants may be used to determine the degree of similarity between the at least one sample and the at least one reference.

In still another embodiment, the present teachings describe a computer readable medium having stored thereon instructions which cause a general purpose computer to perform the steps of: (i) acquiring sequence information relating to at least one sample and to at least one reference for purposes of comparison; (ii) evaluating the sequence information relating to the at least one sample to identify ambiguous bases present within the sample on the basis of the following criteria: (a) scan position differences, (b) peak height ratios, (c) peak area ratios, and (d) base composition; and (iii) evaluating the quality and coverage of the sample sequence information in comparison to the reference sequence information to identify reportable ranges and sequence variants for the sample sequence information.

In a further aspect, the present teachings describe a computer-based system for performing automated sequence evaluation and used to identify associations between a target sample of unknown familial origin with that of at least one reference sample, the system comprising: a database for storing genetic information describing the sequence composition and characteristics for a plurality of nucleotides relating to the mitochondrial genetic makeup of the target sample and at least one reference sample; a program which performs the operations of: assessing the genetic information to identify a degree of ambiguity associated with each of the plurality of nucleotides wherein ambiguous nucleotides are distinguished from unambiguous nucleotides on the basis of: (a) scan position differences, (b) peak height ratios, (c) peak area ratios, and (d) nucleotide compositions; comparing the genetic information and the degree of ambiguity associated with each of the plurality of nucleotides of the target sample and the at least one reference sample to identify a nucleotide signature that provides distinguishing information used to identify sequence similarities and differences between the target sample and the at least one reference sample; and comparing the nucleotide signature of the target sample to that of the at least one reference sample such that substantially identical nucleotide signatures identify the target sample as being of the same familial origin as the at least one reference sample and nucleotide signatures which are not substantially identical identify the target sample as not being of the same familial origin as the at least one reference sample.

Other embodiments of the present teachings provide a system and methods for calculating ambiguous base positions in the genetic sequence reads of mtDNA. Still other embodiments provide systems and methods for real-time monitoring of sequence quality and rapid feedback on the success rate and quality of the sequence data produced.

### BRIEF DESCRIPTION OF THE DRAWINGS

FIG. 1 illustrates an exemplary sequencing and analysis strategy used in mtDNA analysis.

FIG. 2 illustrates an overview of an automated sample processing approach used to associate a sample sequence with that of one or more reference sequences.

FIG. 3 illustrates a system for conducting sequence autoanalysis.

FIG. 4A illustrates block diagram of an mtDNA autoanalysis method used to identify and classify ambiguous and unambiguous bases.

FIG. 4B illustrates an exemplary rule-based analysis approach to distinguish ambiguous and unambiguous bases.

FIG. 4C illustrates an exemplary rule-based approach for quality value assignment.

FIG. 5 entitled Table 1 describes the function performed by various software programs that may be integrated into the autoanalysis approach.

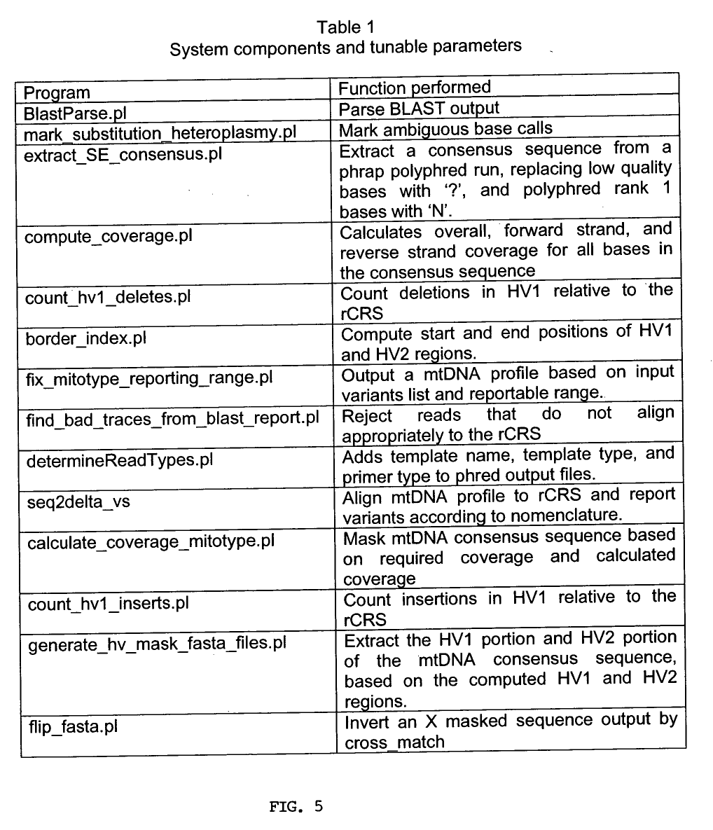

FIG. 6 entitled Table 2 illustrates various validation statistics comparing the use of the autoanalysis methods with conventional manual-based analysis methods.

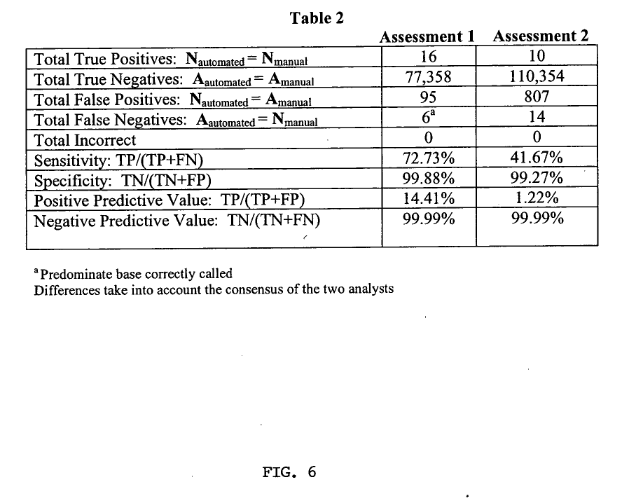

### DETAILED DESCRIPTION OF CERTAIN EMBODIMENTS

The following detailed description of certain embodiments presents various descriptions of specific embodiments of the invention described herein. However, embodiments of the invention can be embodied in a multitude of different ways as defined and covered by the claims. In this description, reference is made to the drawings wherein like parts are designated with like numerals throughout.

Overview

While various embodiments of the present teachings are directed towards automated analysis of sequence information described in the context of sample identification using mtDNA sequencing data; one skilled in the technology will appreciate that the systems and methods described herein may similarly be configured to implement many other types of sequencing applications in addition to mtDNA sequencing. As the mtDNA sequencing system is presented as an illustrative embodiment, the scope of the present teachings is not limited exclusively to this embodiment, but rather includes additional implementations as well.

Terms and Definitions

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

DNA sequencer: an automated electrophoresis detection apparatus used to detect the passage of migrating bands in real time to determine a nucleotide sequence. Examples of automated sequencers include, but are not limited to, sequencers available from Applied Biosystems, Inc (Foster City, Calif.), Pharmacia Biotech. Inc. (Piscataway, N.J.), Li-Cor, Inc. (Lincoln, Nebr.), Molecular Dynamics, Inc. (Sunnyvale, Calif.) and Visible Genetics Inc. (Toronto). Other methods of detection, based on detection of features inherent to the subject molecule, such as detection of light polarization are also possible.

Sequence traces: visual representations of the predominance of a particular nucleotide, or base, at a given position in the nucleotide sequence, as detected on a DNA sequencer, often in the form of multi-color curves (for example sinusoidal or Gaussian peak spread functions). Individual bases may be distinguished by the characteristics of the peaks or curves (e.g. color).

Base calls: the determination of the particular nucleotide, or base, at a given position. The determination can be made either manually or by an automated system using the electrophoretic separation of DNA or RNA fragments of a sequencing reaction.

Unambiguous base calls: A (adenine), C (cytosine), G (guanine), and T (thymine) for DNA, or U (uracil) for RNA.

Ambiguous base calls: A base call could not be reliably made. Ambiguous base calls are designated by an N. Ambiguous base calls may be identified for sequence several reasons, including for example: 1) low quality sequence information, 2) insufficient coverage or mapping, 3) non-unique bases for a selected position (e.g. mixed-bases), and 4) heteroplasmy. The presence of heteroplasmy (resulting from a mutation in a fraction at the mtDNA from one person) at a site is often quite specific to an individual, and therefore valuable in making identifications. However, it can also vary from tissue to tissue and can be misinterpreted as high background or contamination of the sample. In sequence alignment and mtDNA profile comparison, N can match any base (A, T, C, or G).

Quality values: a measurement or quantitation of the accuracy or confidence of a base call.

Consensus sequence: a sequence resulting from the compilation, or assembly, of sequence traces for various fragment of the sequence, wherein the consensus sequence reflects the most frequent base call at each scan position.

True Positive (TP)—In assessing the performance of the system and methods as described herein and used as a designation for a base identified by the automated data analysis system as ambiguous and manually verified to be ambiguous. For example, the automated system may call a base position an N and manual review may also call it an N. (e.g. N_{automated}=Nₘₐₙᵤₐₗ)

True Negative (TN)—In assessing the performance of the system and methods as described herein and used as a designation for a base identified by the automated data analysis system as unambiguous (A, C, G, or T) and manual verified review indicates the base position as unambiguous (A, C, G, or T). (e.g. A_{automated}=Aₘₐₙᵤₐₗ)

False Positive (FP)—In assessing the performance of the system and methods as described herein and used as a designation for a base identified by the automated data analysis system as ambiguous and manual review verified to be unambiguous. For example, the automated system called a base position an N and manual verified review indicates the base position as unambiguous (A, C, G, or T). (N_{automated}=Aₘₐₙᵤₐₗ)

False Negative (FN)—In assessing the performance of the system and methods as described herein and used as a designation for a base identified by the automated data analysis system as unambiguous and determined to be ambiguous by manual review. For example, the automated system may call a base position as unambiguous (A, C, G, or T) and manual review indicates the base position as unambiguous (N). (A_{automated}=Nₘₐₙᵤₐₗ)

Introduction

Forensic analysis or molecular fingerprinting based on identification of DNA sequences represents an important tool for identification and familial association of biological samples and genetic materials of uncertain origin. Some of the more common sources of genetic material that may be used in conjunction with these identification techniques include, but are not limited to: skin, hair, saliva, semen, tissue, bone, and blood. When performing sequence-based identification analysis, preservation of sequence integrity is important in order to assure proper sample identification. Unfortunately, this is not always possible and as a result sequencing analysis must sometimes be conducted using partially degraded or contaminated samples. Recently, mtDNA analysis techniques have established themselves as preferable over nuclear DNA analysis in certain instances due in part to the degradative tolerance and relative high copy number of mtDNA.

As an example of how mtDNA analysis may be performed in a forensic context, an investigator typically compares mtDNA obtained from an unknown sample (e.g. skeletal remains or other genetic samples) with mtDNA isolated from at least one presumed maternal relative. Mitochondria, organelles which supply energy to many different cell types—including bone cells—contain their own DNA. In humans, there are approximately 16,500 base pairs of DNA associated with the mitochondrial genome. Of this, two regions contain significant base sequence variation that provides distinguishable mitochondrial profiles useful in developing molecular fingerprints that can assist in identifying individuals or genetic samples. In humans these two regions of the mitochondrial genome are referred to as Hypervariable Region I (HVI) and Hypervariable Region II (HVII) and comprise relatively small sequence stretches as compared to the approximately 3.5 billion bases that make up the human genome.

Sample identifications and associations are generally based on the observation that mtDNA isolated from a genetic sample for a selected individual and from maternal relatives have substantially the same sequence of bases in the hypervariable regions of the mitochondrial genome, and these same sequences will typically vary from mtDNA sequences isolated from genetic samples obtained from unrelated individuals. By obtaining genetic samples from presumed or candidate maternal relatives associated with a person from which a biological sample is derived, an investigator can compare the sequence patterns for the mtDNA in the collected samples with mtDNA extracted from an unidentified biological or genetic sample of questionable origin to aid in identification. For example, if the DNA sequence patterns do not match at two or more positions, the investigator may conclude that the origin of the samples exclude a familial linkage.

Conventional, methods for performing such analysis are labor intensive and error-prone due to the requisite dependence on human evaluation of the sequencing data. Furthermore, in large scale applications were many samples may be desirably analyzed, difficulties are often encountered in terms of increased complexity of data analysis and extended times required to complete the analysis.

A further problem encountered when performing these types of analysis is that there is an inherent uncertainty that often arises during base-calling and sequence evaluation. Uncertainties of this type arise for many reasons and are reflected in “quality value” assessments that may be associated with each base within a sequence or with a portion or the whole of the sequence itself. Quality value assessment and/or evaluation of uncertainties and ambiguities in base-calling are integral to the sequence analysis and may confound sample identification or familial association. Furthermore, conventional methods for evaluating quality values and base-call ambiguities when performed through software-driven interpretation methods are error-prone and may not provide optimal results. Conversely, detailed investigator review of this information is time consuming, laborious, and may be impractical in instances where large amounts of information must be processed.

Conventional base-calling programs including Phred (Ewing et al. Genome Res. March 1998; 8(3):175-85 and PolyPhred (Nickerson et al. Nucleic Acids Res. Jul. 15, 1997; 25(14):2745-51 have been previously described for purposes of base-identification (e.g. base-calling) and genotype of single nucleotide substitutions. For example, the Phred base-caller uses a four-phase procedure to determine a sequence of base-calls from a processed trace file, electropherogram, or chromatogram data. Briefly described, this methodology is generally directed towards base identification using predicted and observed peak positions derived from the input data. Error estimation during Phred base-calling is necessary to account for misinterpretation of peaks and to discriminate errors from correct base-calls. Several parameters are involved in the process and include peak spacing determinations, uncalled/called ratios, and peak resolution characteristics. In general, each of these parameters attempts to relate information contained in selected windows of multiple peaks. Error probability calculations obtained from Phred may relate to base-call confidence interpreted as quality values which typically span a wide range (for example from 0-50).

A problem observed with application of the Phred base-calling approach is that systematic biases may be introduced resulting in over-predicted or under-predicted error-rates that may undesirably affect the quality value determination and usability in terms of distinguishing and validly identifying both ambiguous and unambiguous bases present within a sequence. Furthermore, this approach is sensitive to variations in the quality of the trace or raw sequence data and, as a consequence, it can be extremely difficult to reduce the error rate to a sufficient level to infer highly accurate sequence information. Even when using high-quality trace or raw sequence data, conventional base-calling approaches encounter difficulties when attempting to identify and resolve base compressions (e.g. GG and CC).

The methods described by the present teachings address these issues of uncertainty and error prone base-calling by providing an improved method for evaluating sequence data and information that aid in the identification and confidence assessment. In certain instances, base-calls that would be characterized by a low quality value using conventional means may be evaluated/re-evaluated using the methods described by the present teachings with an improvement in base-calling confidence and ambiguity resolution. In certain embodiments, this improved base-calling discrimination may be attributed to a novel analytical paradigm in which various characteristics of the input sequence data are used to classify each base-call as ambiguous or unambiguous. Base-calls that are identified as being ambiguous may further be subject to more rigorous treatment to further resolve ambiguity. Additionally, quality value estimates may be adjusted according to various criteria to narrow the output range of quality values thereby facilitating the characterization of base-call quality and certainty.

In various embodiments the present teachings desirably improve the efficiency and accuracy of sequence analysis and sample identification by providing an automated approach to data acquisition and evaluation. In particular, the disclosed methods may be adapted to sequence identification techniques such as forensic analysis to provide an automated approach to data interpretation thereby reducing the conventional limitation of detailed investigator review. Implementation of these automated methods desirably provides a means by which to improve the speed and accuracy of the analysis as compared to conventional methods and may be used to improve identification throughput especially in large or complex sequencing projects. Furthermore, the methods described herein may be desirably implemented in an automated manner and coded in software or hardware while still preserving the ability to effectively analyze and interpret quality value information.

In another aspect, the present teachings describe a method by which quality value information can be quickly and efficiently assessed to identify base-calls as having a threshold degree of certainty associated with them or as ambiguous. This manner of base-call analysis is particularly useful in determining which base-calls may be subsequently used in sample identification and comparison to other sample sequences. In various embodiments, this manner of analysis provides a useful “pass/fail” criterion for assessing base-calling quality and improves performance and efficiency when comparing sequences from different origins.

It will be appreciated that while the disclosed methods are described in the context of performing mtDNA-based sequence analysis, other types of sequence-based analytical methods including nuclear DNA analysis may be adapted for use with the present teachings to provide means for increasing their sequence analysis automation capabilities.

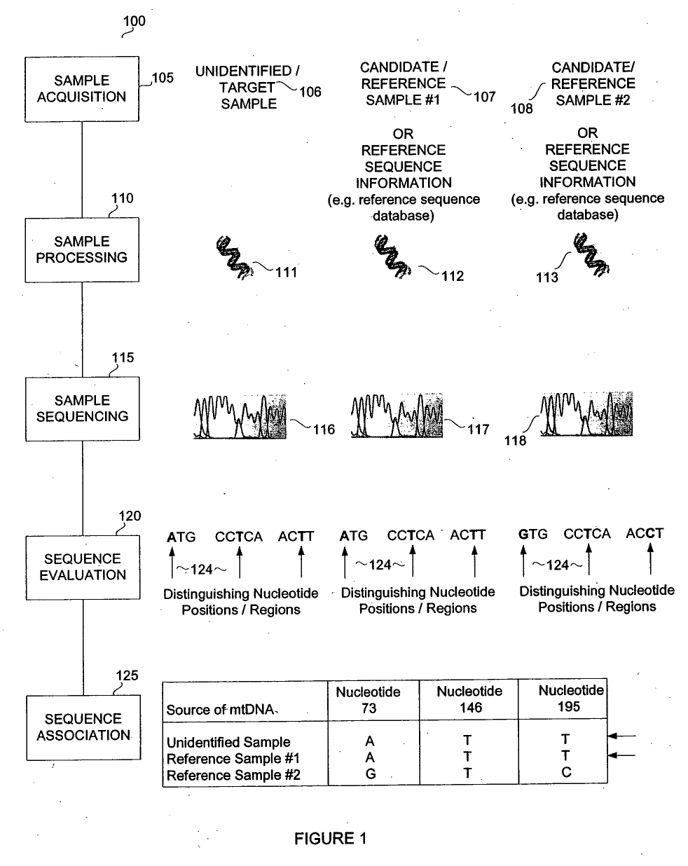

FIG. 1 illustrates a simplified mtDNA sequencing strategy 100 used to identify familial associations between a sample of unknown familial origin with that of at least one familial reference sample. As will be described in greater detail herein below this analysis procedure is desirably adapted to be partially or fully automated through the use of the methods described by the present teachings. The method 100 commences in state 105 with the acquisition of the unidentified or target sample 106 and at least one candidate or reference samples 107, 108. As previously indicated the samples may be of various different types, including but not limited to tissue, skin, blood, bone, etc. Following sample acquisition, sample processing may take place in state 110 to isolate genetic material 111, 112, 113 of interest from each sample. In various embodiments, the genetic material is representative of isolated DNA or RNA that is present in a form suitable for further processing. Sample processing in state 110 may involve numerous procedures including for example: isolation and/or purification of the genetic material, amplification of the genetic material, incorporation of suitable markers or labels used in sequencing, to name a few. In general, these procedures prepare the genetic material from each sample for subsequent sequencing analysis in state 115 wherein raw sequence information 116, 117, 118 is obtained by conventional sequencing methods. In one aspect, the raw sequence information comprises information including: electropherogram/sequence traces, information describing peak characteristics such as peak height/width/areas, putative base identifications/base calls, quality value assessments, scan positions, and other information.

The information obtained from the sequencing analysis is processed in state 120 to perform sequence evaluation operations wherein one or more selected regions of the sequence associated with the genetic material from each sample is used for purposes of associating/distinguishing the unidentified or target sample 106 and the candidate or reference samples 107, 108. In one aspect, this analysis comprises identifying certain nucleotides or nucleotide positions that display some degree of sequence variation between the unidentified or target sample 106 and the candidate or reference samples 107, 108. For example, certain nucleotide positions 124 in the base sequences may be flagged or identified as providing distinguishing information useful in the sample analysis. These nucleotide positions 124 are evaluated across each sample to generate a collection of information that may be used to determine any association between the unidentified/target sample 106 and the candidate or reference samples 107, 108 in state 125.

As shown by way of illustration in FIG. 1, a plurality of discrete nucleotide positions 124 taken together form an identifying base sequence or signature that may be used to associate and distinguish the samples 106, 107, 108. Here the base signature “ATT” present in both the unidentified sample 106 and the reference sample 107 indicates a potential commonality of origin between the two. Conversely, the differing base signature “GTC” of the reference sample 108 suggests a lack of commonality with the unidentified sample 107. In this manner, the sample associations may be performed and the origin of the unidentified sample determined.

In performing the aforementioned sequence evaluation and sequence association analysis care must be taken when selecting base positions that will be used to associate and distinguish the samples. For example, it is important to determine which positions have base calls that are sufficiently ambiguous such that they should be excluded from use in associating the samples. Ambiguous base-calls (represented in part by a low quality or confidence values) may contribute to decreased sample identification efficiency and may lead to errors in analysis and identification. Thus it is desirable to avoid using such ambiguous bases when practical to improve identification accuracy.

Furthermore, utilization of ambiguous base information typically requires more detailed analysis either in terms of computational complexity or volume of information to be processed. For example, if a significant percentage of bases are utilized which possess a high degree of ambiguity more bases are needed to increase the likelihood of accurate identification. In various embodiments, it is an object of the present teachings to assess and exclude ambiguous bases from the identification analysis so as to increase the reliability of the match of an unknown sequence to the reference and further to improve the speed of the analysis. As will be described in greater detail hereinbelow, the methods for ambiguous base determination are predicated upon a relatively straightforward set of rules that are amenable to coding in software or hardware and provide a means by which to perform the analysis in an automated manner without a significant requirement for investigator interpretation or review.

In one aspect, the methods described herein are directed towards the processing of a variety of information associated with a genetic sample sequencing run in such a manner so as to distinguish ambiguous bases from other bases which may be more suitable for the type of comparisons used in sample identification. Distinguishing the bases in this manner may improve the efficiency and accuracy of sample identification using less processing time with greater accuracy than is routinely achieved by conventional methods. These features are particularly useful in the context of improving the performance and capabilities of high-throughput analysis.

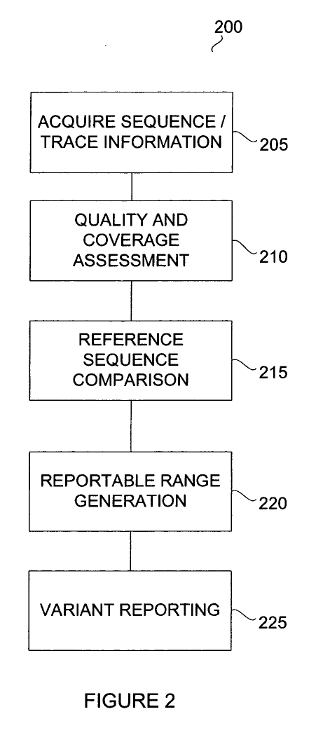

FIG. 2 presents an overview of the automated sample processing approach 200 described by the present teachings. In various embodiments, the system and methods described herein may be desirably used to process both large and small numbers of samples and in particular may be used to efficiently perform mtDNA analysis for sample volumes on the order of 40,000 samples or more. Conventional means by which to conduct analyses of this magnitude are impractical for reasons including the length of time required to complete the analysis as well as the degree to which investigator review is needed to insure accuracy.

In one aspect, the approach 200 incorporates a plurality of software functionalities or programs that are used to provide reliable mtDNA profiles for assessing the data quality and performance of laboratory processing methods associated with forensic sequence analysis. One manner of assessment in this context comprises generating a plurality of provisional profiles that may be used for the comparison of at least one victim, unknown, or target sample with one or more reference samples.

As shown in FIG. 2, the approach 200 commences in state 205 wherein information acquisition takes place. The information utilized in the autoanalysis approach comprises information generated according to known methods for sequencing of desired regions of target genetic material. Information utilized during autoanalysis may include experimentally determined base sequence data for each sample and may further comprise both base-call information as well as other sequence information including quality value/confidence assessments, consensus/alignment information, raw trace or electropherogram information, and other data associated sequence alignments and output.

The aforementioned sequence information or parts thereof, serve as input for autoscoring analysis and in state 210 the quality and coverage of the experimentally obtained sequence data may be assessed. The resulting information may then be compared against reference sequence information in state 215. The reference sequence information comprises known sequence information that has been previously identified and validated and may include archived sequence information obtained for example from the revised Cambridge Reference Sequence database (rCRS). The comparison of sequence information and evaluation of quality and coverage of the data is subsequently followed, in state 220, by the generation of reportable ranges that are defined for the experimentally obtained sequence data. This information is further used as a criterion for identification of sequence variants and further to associate the target sequence with suitable reference sequences which may then be output in state 225.

As will be appreciated by one of skill in the art, the above-described steps of the method 200 are not necessarily limited exclusively to the order described and certain steps may be re-arranged as desired or convenient from the analytical standpoint. For example, in certain embodiments the comparison of reference sequence information performed in state 215 may precede the quality and coverage assessment performed in state 210.

As will be described in greater detail hereinbelow, the autoscoring method implemented in this approach may be used to process DNA sequence reads or traces obtained from one or more samples in an automated manner replacing manual data analysis and review of mtDNA sequence data. In particular, this approach may augment or replace manual review in the context of analysis of coverage requirements and detection of mixtures and heteroplasmic sites with a suitable means for conducting these analyses in a semi-automated or fully-automated manner.

In certain embodiments, the automated methods may further be characterized as performing a number of operations that assist in comparing target genetic information to reference genetic information by identifying ambiguous and variant bases. As previously described, ambiguous base identification according to the present teachings may improve the speed and efficiency of the identification analysis by avoiding use of sequence information which may be less useful for comparison purposes. Furthermore, these methods seek to increase the amount of sequence information that possesses a relatively high degree of base-call confidence thus improving the accuracy of the comparison.

In various embodiments, the autoscoring methods described by the present teachings may be incorporated into existing hardware/software-based analysis packages/platforms and need not necessarily be exclusively limited to mtDNA comparison analysis. For example, ambiguous base determination according to the autoscoring approach may be useful in other sequencing operations and the results integrated into consensus analysis approaches for routine nucleotide strand sequencing (e.g. DNA or RNA sequencing). Furthermore, the autoscoring methods may be used in conjunction with existing sequence information and data to aid in the identification of ambiguous bases. The methods described herein are also flexible in that they may be modified to accommodate other rule sets useful in other contexts and analysis approaches.

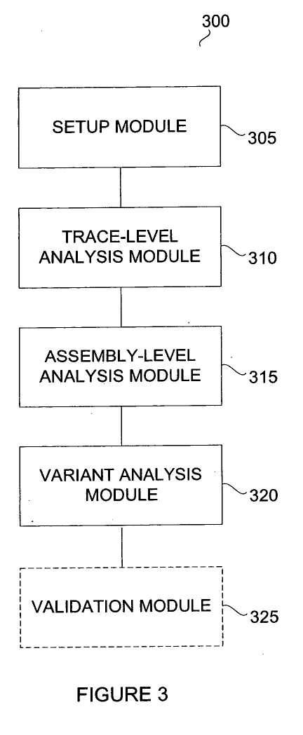

FIG. 3 illustrates a system 300 used to conduct sequence autoanalysis according to the aforementioned approach. In various embodiments, the automated data analysis system 300 operates by preparing, analyzing, and processing trace files, or raw sequence data. The raw sequence data, or sequence traces, can be obtained, for example, from a DNA sequencer, which generates sequence data using fluorescent-based capillary electrophoresis. In some embodiments, the raw sequence data is analyzed by the automated data analysis system 300 in the context of known standard sequence information and operator defined rules. Examples of known standards include, but are not limited to, the original Cambridge Reference Sequence (CRS) (Anderson et al., Nature (1981)) and the revised Cambridge Reference Sequence (rCRS) (Andrews et al., Nat. Genet. (1999)). Examples of operator defined rules include, for example, rules regarding thresholds for determining sequence quality, ambiguous base positions, and coverage requirements for reporting a result.

The setup module 305 comprises the data entry component of the system 300 and provides means for receiving and checking the validity of the input parameters and information. In certain embodiments, this component includes functionality for performing operations including building the directory structure for output files, reading input trace files and information, and generating sequence and quality value data files. The setup module may also perform various pre-processing functions including data formatting and processing according to various input parameters. The input parameters provide the system with information about which samples are to be processed and several aspects of quality and coverage. In certain embodiments, the input parameters may be user specific and can be converted from a format recognized by the automated sequencer to a format recognized by the automated data analysis system before being read, for example, from AB1 format to SCF format.

User-defined input parameters include, but are not limited to the following parameters: the maximum ratio for transition heteroplasmy; the maximum difference between scan positions for transition heteroplasmy, the minimum ratio for a mixture, the maximum difference between scan positions for a mixture, the minimum number of bases for a mixed base run, the minimum number of bases for a clean base run, the maximum quality of a low quality base, the number of starting bases that get a limited quality value, the minimum number of bases for a homopolymer run, and the minimum number of unambiguous bases (A, C, T, or G) for a homopolymer run. The user may further define the parameters in a configuration that will mimic certain rules or combinations of rules used in manual analysis of the data. The following parameters and ranges are described in the context of performing mtDNA analysis using the automated system and methods described herein. It will be appreciated that automated analysis may not necessarily be limited exclusively to these parameters and ranges. Consequently, various additions, substitutions, and modifications to the user-defined parameters may be observed without departing from the scope of the present teachings.

For example, the minimum ratio for transition heteroplasmy can be configured as a user parameter having a range from approximately 0.05 to about 0.5, preferably between approximately 0.25 to about 0.5, and more preferably from approximately 0.3 to about 0.4.

Likewise, the maximum difference in scan position for transition heteroplasmy can be configured as a user parameter having a range from about 1 to about 6, preferably between about 2 to about 5, and more preferably from about 3 to about 4.

The minimum ratio for a mixture can be defined from about 0.05 to about 0.5, preferably between about 0.20 to about 0.5, and more preferably from about 0.3 to about 0.4.

The maximum difference in scan position for a mixture can be defined, for example, from about 1 to about 6, preferably between about 2 to about 5, and more preferably from about 3 to about 4.

The minimum number of bases for a mixed base run can be defined, for example, from about 1 to about 10, preferably between about 3 to about 8, and more preferably from about 5 to about 7.

The minimum number of bases for a clean base run can be defined, for example, from about 1 to about 10, preferably between about 3 to about 8, and more preferably from about 5 to about 7.

The maximum quality of a low quality base can be defined, for example, from about 7 to about 30, preferably between about 10 to about 20, and more preferably from about 14 to about 18.

The maximum quality of a low quality base can be defined, for example, from about 10 to about 35, preferably between about 20 to about 30, and more preferably from about 25 to about 28.

The number of starting bases that have a limited quality value can be defined, for example, from about 2 to about 25, preferably between about 7 to about 15, and more preferably from about 10 to about 13.

The minimum number of bases for a homopolymer run can be defined, for example, from about 4 to about 25, preferably between about 7 to about 15, and more preferably from about 10 to about 13.

The minimum number of unambiguous bases for a homopolymer run can be defined, for example, from about 3 to about 24, preferably between about 6 to about 14, and more preferably from about 9 to about 12.

The input data may include, but are not limited to, data concerning the peak area, peak height, and scan-position information for the major and minor peaks identified and called by an automated sequencer. The input data may further include, for example, data concerning the base call and corresponding quality value information calculated from an electropherogram file.

In certain embodiments, the setup module may provide a method for verifying that the input parameters are within an expected range.

The step of building the directory structure for output files may include, for example, the steps of creating sub-directories and transferring relevant trace files into those directories from the trace archive.

In certain embodiments, the setup module performs various functions after receiving input parameters and data, such as producing additional base-calls, quality values, and other information useful in evaluating peak characteristics and trace data associated with input sample sequence data. In this regard, known analysis applications may be integrated into the setup module to provide the desired functionality. For example, the functionality of the phred-phrap (PP) software analysis tool (University of Washington, WA) may be integrated into the setup module (and other modules) to facilitate analysis as will be described in greater detail hereinbelow.

The trace-level analysis module 310 provides a means for preparing the trace files for assembly. In certain embodiments, this module may include functionality for removing poor quality trace files from further processing; analyzing the remaining trace files; and trimming the PCR primer sequences from the trace files.

In various embodiments, poor quality trace files are removed from further processing by comparing the trace file to the reference sequence data (e.g., rCRS) to ensure that a single region of sequence similarity exists between the trace file and the reference sequence data. In alternative embodiments, the step of removing poor quality trace file from further processing may include, for example, verifying that the trace file has met user-defined quality thresholds. Examples of user-defined quality thresholds include, but are not limited to, minimum thresholds for length and percent identity of the alignment.

In some embodiments, analysis of the remaining trace files further comprises functionality for reading the trace files; generating sequence, quality value, and peak height, peak area, and scan position data files; and marking bases with a base identifier. The base identifier may include a base identifier for unambiguous bases and a base identifier for ambiguous bases. The base identifier may further include an identifier for ambiguous bases, a base identifier for unambiguous bases, and a base identifier for bases following a homopolymeric stretch. The base identifier for ambiguous bases can further distinguish between ambiguous bases that are caused by background noise, ambiguous bases caused by heteroplasmy, and ambiguous bases caused by mixed samples or contamination. The base identifiers for unambiguous bases can be a single letter corresponding to the first letter of the nucleotide, that is A, C, T, or G. The base identifier for ambiguous bases can be a single letter, for example, the letter “N.” The base identifier for bases following a homopolymeric stretch can similarly be a single letter, such as, the letter “X.” Additionally, other base identifiers may be utilized, for example, mixed-base identifiers corresponding to ambiguous bases of selected compositions according to the IUPAC nomenclature (e.g., A or G=R, C or T=Y. etc.).

Further, in some embodiments, the quality value may be adjusted based on user defined input parameters. The user-defined parameters can be chosen to approximate certain operations associated with manual analysis of sequence traces. Further, the adjustment of the quality values can be temporary or artificial to aid in the assembly-level analysis module. For example, quality values of unambiguous bases can be adjusted to a maximum quality value such that the quality values of unambiguous bases are not prejudicial in the assembly of the consensus sequence.

The assembly-level analysis module 315 provides a means for preparing a consensus sequence. In some embodiments, this module 315 may include functionality for compiling overlapping trace files; assembling a consensus sequence; and comparing the consensus sequence to a known standard sequence or data set.

This module may further include functionality for performing additional base analysis on the assembled sequence. For example, a proofreading system can identify potentially mixed base sites or discrepancies between overlapping sequence traces. The discrepancies between overlapping sequences can, in some embodiments, be marked ambiguous.

In other embodiments, the module 315 may perform reverse complementing the sequence in L-strand orientation for comparison with the known standard, according to the nomenclature used in the forensic community. Further, the step of comparing the consensus sequence to a known standard can include recording the differences between the consensus sequence and a known standard sequence or data set (e.g., CRS or rCRS).

In other embodiments, the module 315 compares the coverage of the consensus sequence to the user-defined threshold for sequence coverage, and generates a new consensus sequence that masks bases that do not meet user-defined thresholds. Examples of user-defined thresholds, include, but are not limited to, the location and size of permitted single-stranded coverage regions. In the context of mtDNA analysis, due to the frequently encountered difficulty of sequencing through the poly-cytosine segments of HVI and HVII of mtDNA, a short region around each poly-cytosine segment may be permitted to be single-stranded covered. The resulting consensus sequence can then be re-aligned with the known standard to determine variations.

The variant analysis module 320 provides functionality for preparing an mtDNA profile. In some embodiments, this module 320 may include functionality for performing the steps of determining the reportable range of the consensus sequence; and formatting the final profile based on the variations between reportable range of the consensus sequence and a known standard or reference sequence or data set. Determining the reportable range of a consensus sequence can include operations including trimming the edges of the consensus sequence until the percentage of ambiguous bases over a specified number of bases is below a user-defined threshold. Formatting the final profile can include operations including identifying variants and storing the variants to a text file or database. The variant information may include, for example, reference sequence positions and base calls for all differences with respect to the standard or reference data. For example, a deviation at position 263, from an A in the standard to a G in the sample might be recorded as “263 G.” Variant reports may then be compared to a reference data set for the purpose of validation 325. Alternatively, variant reports for multiple samples can be compared to one another to exclude potential or candidate matches. In certain embodiments, validation may be used to assess certain parameters to evaluate the accuracy or performance of the analysis. This information may further be compared to a reference standard of manually defined mtDNA profiles and may include definition of reportable ranges, accuracy of base-calls, and accuracy of identification of insertions/deletions.

Sequence Analysis

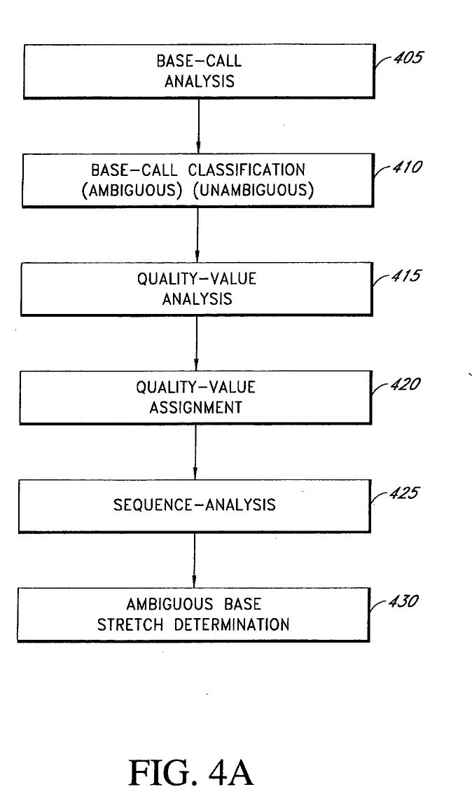

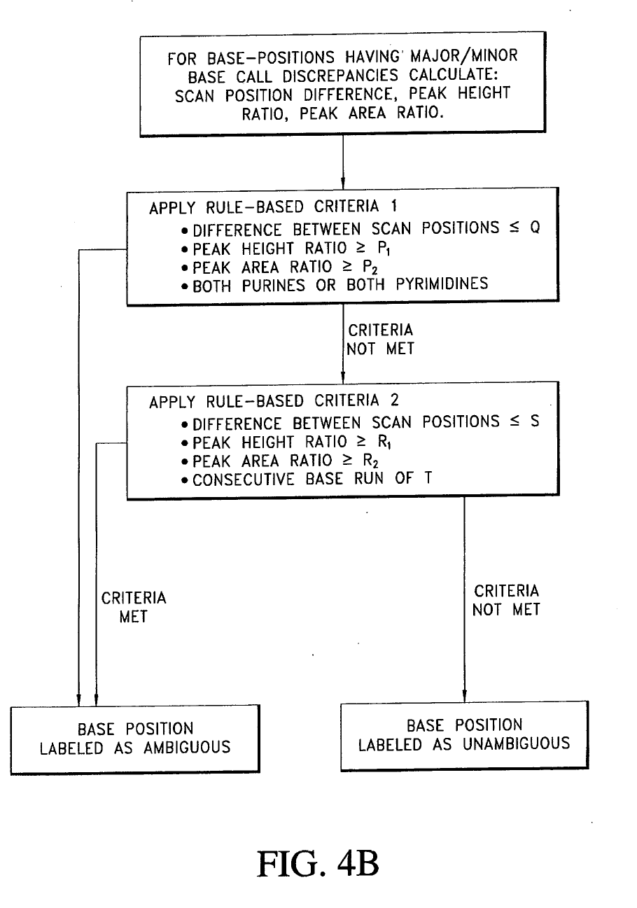

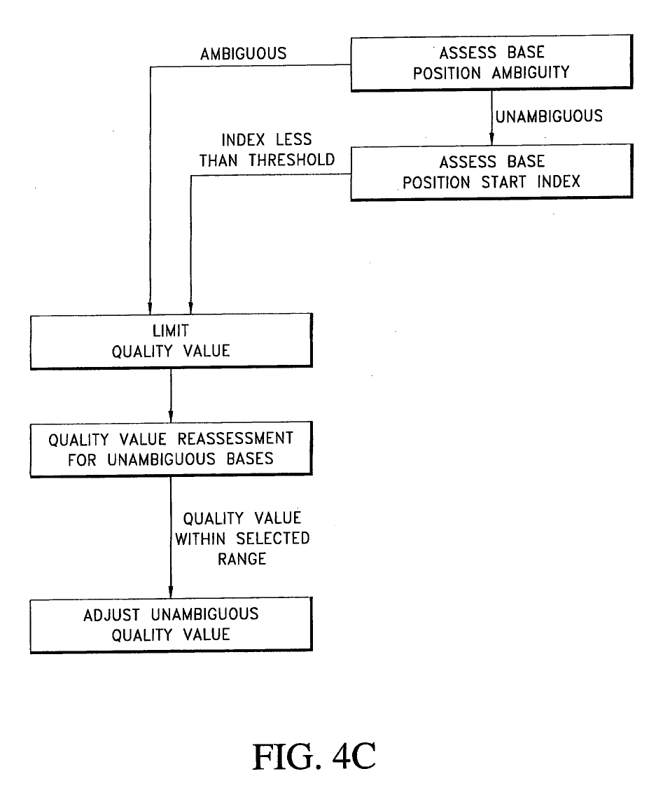

FIG. 4A illustrates a functional block diagram of a method 400 for analyzing trace files associated with mtDNA analysis. In various embodiments, this method 400 may be desirably utilized to assess trace file and sequence information for samples of interest and to identify/distinguish base-call information as ambiguous or unambiguous. The method 400 commences in state 405 wherein a base-call analysis is performed for each base of the input or sample sequence. Using information including peak area, peak height, and scan-position, bases are desirably identified as ambiguous or unambiguous according to a rule-based criteria set. In performing this analysis 405, a determination is made to identify base-position discrepancies. For example, if the major base is A, C, G, or T and the minor base is A, C, G, or T and the major base is not the same as the minor base, then the following values may be calculated: (1) the distance between scan-positions of the minor base and major base; (2) the peak height ratio between the minor base and major base; and (3) the peak area ratio between the minor base and major base.

Subsequently, the aforementioned calculations may be used in the following manner to label bases as ambiguous or unambiguous. In general, a series of pre-selected/user identified rules are established based on the criteria of scan position differences, peak height ratios, and peak area ratios to classify the ambiguity of each base position. These rules desirably utilize information readily obtainable from the sequence/trace file data and are amenable to inclusion in a software-based analysis approach alleviating the need for manual evaluation. Furthermore, the rules themselves generally do not involve highly complex calculations and can be performed without undo computational overhead thereby improving the performance of the analysis.

FIG. 4B illustrates an exemplary rule-based analysis performed in state 405 that results in base-call classification in state 410. If the distance between scan-positions of the minor base and major base is less than or equal to a user-identified threshold associated with transition heteroplasmy (Maximum_scan_delta_for_transition_heteroplasmy) and the peak height ratio between the minor base and major base is greater than or equal to a user-identified threshold associated with transition heteroplasmy (Minimum_ratio_for_transition_heteroplasmy) and the peak area ratio between the minor base and major base is greater than or equal to a user-identified threshold associated with transition heteroplasmy (Minimum_ratio_for_transition_heteroplasmy) and the major base and minor base are both purines (A,G) or are both pyrimidines (C,T) then the base position may be marked or identified as ambiguous.

For those base positions not meeting this criteria, if the distance between scan-positions of the minor base and major base is less than or equal to a user-identified threshold associated with a mixed base (Maximum_scan_delta_for_mixture) and the peak height ratio between the minor base and major base is greater than or equal to a user-identified threshold associated with a mixed base (Minimum_ratio_for_mixture) and the peak area ratio between the minor base and major base is greater than or equal to a user-identified threshold associated with a mixed base (Minimum_ratio_for_mixture) and there has been a consecutive run of a user-identified number of such bases (Minimum_number_of_bases_for_mixed_base_run), then the run of base-calls is labeled as ambiguous.

As a final criteria for distinguishing between ambiguous and unambiguous base call runs; if the major base is an N, and there has been a consecutive run corresponding to the threshold above (Minimum_number_of_bases_for_mixed_base_run), then the run of such bases is labeled as ambiguous; Alternatively, if the major base is an A, C, T, G, and not mixed and there has been a consecutive run corresponding to a threshold associated with a clean base run (Minimum_number_of_bases_for_clean_base_run), then the run of base-calls is labeled as unambiguous.

Referring again to FIG. 4A, a quality value analysis is performed following identification of ambiguous and unambiguous bases in state 415 and quality values are assigned/adjusted in state 420. The quality value analysis is directed towards establishing the confidence or accuracy of each base call and further adjusts the quality values to facilitate analysis. In one aspect, quality value assessment and assignment is directed towards adjusting the quality values for base positions identified as ambiguous or unambiguous to simplify subsequent processing and mtDNA sample identification. Furthermore, the adjusted quality values serve to further identify ambiguous bases or base stretches that may be less useful in subsequent analysis thereby improving the efficiency with which mtDNA analysis can be performed.

FIG. 4C illustrates an exemplary rule-based analysis performed in state 415 that results in quality value assignment in state 420. For each nucleotide position, the base call and corresponding quality value data calculated from an electropherogram file may be modified, if necessary based on the analysis of data from previous steps, and from analysis of homopolymer regions. Briefly described, if a base was previously labeled as ambiguous, the notation of the base is changed to an ‘N’, and the quality value is limited to a maximum user indicated value (Maximum_quality_of_low_quality_base). For unambiguous bases, if the index of the base is less than or equal to a pre-selected quantity (Number_starting_bases_that_get_limited_quality), then the base quality value is limited to a maximum user selected value (Maximum_quality_of_low_quality_base). Furthermore, for unambiguous base-calls, if the quality value is determined to be greater than 10, but less than a user specified value (Minimum_quality_of_high_quality_base), then the quality value is set to be equal to the user specified value (Minimum_quality_of_high_quality_base).

In certain embodiments, if there has been a consecutive run of unambiguous bases that are identical and ambiguous bases of length greater than or equal to a user specified value (Minimum_number_of_bases_for_homopolymer_run), and in this consecutive run, the number of identical unambiguous bases is greater than or equal to a user specified value (Minimum_number_of_non_n_bases_for_homopolymer_run), then each of the bases subsequent to the identified run is changed to ‘X’, and the quality value of all bases after the run is limited to a maximum value determined by the user specified value corresponding to the Maximum_quality_of_low_quality_base.

Components of the Automated Data Analysis System

As previously indicated, in certain embodiments, the automated data analysis system may include software programs and algorithms developed by Applera Corporation. For example, some embodiments of the system include the following programs: BlastParse.pl; find_bad_traces_from_blast_report.pl; mark_substitution_heteroplasmy.pl; determineReadTypes.pl; extract_SE_consensus.pl; seq2delta_vs; compute_coverage.pl; calculate_coverage_mitotype.pl; count_hv1_deletes.pl; count_hv1_inserts.pl; border_index.pl; generate_hv_mask_fasta_files.pl; fix_mitotype_reporting_range.pl; flip_fasta.pl. A brief description of each of these programs is provided in Table 1.

Further, in some embodiments, the automated data analysis system may also utilize the following publicly available programs including convert_trace—converts trace files from AB1 format to SCF format; phred—reads trace files and generates sequence and quality value data; phd2fasta—converts phred output files to fasta format; phrap—performs assembly of sequence data using multiple sequence alignment; cross_match—performs sequence comparison using sequence alignment; polyphred—analyzes phrap files and identifies those base positions producing two (2) or more fluorescent signals; formatdb—formats sequence data for use as input to blastall; blastall—performs sequence similarity search of one query sequence against a database of subject sequences (formatted by formatdb); bl2seq—performs sequence similarity comparison of one query sequence against one subject sequence.

### EXAMPLES

The following examples are offered by way of illustration and not by way of limitation. The examples are provided so as to provide those of ordinary skill in the art with a complete disclosure and description of how to develop and use the methods of the present teachings and are not intended to limit their scope. The disclosures of all citations in the specification are expressly incorporated herein by reference.

Several parameters of accuracy have been examined to assess the performance of the automated data analysis system as compared to a reference standard of manually defined mtDNA profiles. These include definition of reportable range, accuracy of ACGT base cells, accuracy of ambiguous (N) base cells, and accuracy of insertion/deletion large cells.

To determine the sensitivity and specificity of the automated data analysis system in calling ambiguous and unambiguous bases, automated data analysis system-generated mtDNA profiles were compared with profiles generated by the consensus of two (2) manual reviews.

### Example 1

Sample Selection and Processing

Seventy-five (75) test samples provided by the City of New York Office of Chief Medical Examiner and seventy-five (75) bone samples from Bode Technology Group (Springfield, Va.) were included in the study. The 150 samples were amplified, sequenced, and analyzed using automated capillary electrophoresis according to standard procedures for mitochondrial DNA processing.

Data Analysis

The sequence traces for each sample were first manually analyzed by two (2) analysts independently and compared to the rCRS. For each sample, a consensus report of variants when compared to the rCRS was generated.

The same set of traces was then processed by the automated data analysis system according to the procedure described above. A side-by-side comparison of the manual analysis and automated results was performed. All differences between manual review consensus and automated calls were counted. The numbers of true positive (TP), false positive (FP), true negative (TN), and false negative (FN) bases were determined. The sensitivity, specificity, positive predictive value, and negative predictive value of automated data analysis system were then calculated based on the formulae described below.

Formulae

Sensitivity—TP/(TP+FN)—this value indicates the relative ability of the automated data analysis system to accurately call ambiguous positions in a sample.

Specificity—TN/(TN+FP)—this value indicates the relative ability of the automated data analysis system to accurately call unambiguous positions in a sample.

Positive Predictive Value—TP/(TP+FP)—this value indicates the likelihood that a position called by the automated data analysis system as ambiguous is actually ambiguous.

Negative Predictive Value—TN/(TN+FN)—this value indicates the likelihood that a position called by the automated data analysis system as unambiguous is actually unambiguous.

Results

As shown in Table 2, the false negative results indicated that the system provided 99.88% specificity and 99.99% negative predictive value on the test sample sets. The sensitivity evaluation indicated that the ability of the system to accurately identify ambiguous positions is reasonable. When comparing the number of false positives versus the number of false negatives, and the positive predictive value, this analysis demonstrates that it would be more likely for the automated data analysis system to call an unambiguous base as ambiguous than vice versa.

### Example 2

Sample Selection and Processing

One hundred (100) samples with all associated trace files from the U.S. Department of Justice FBI's Laboratory Division “mtDNA Population Database” were provided for analysis by the automated data analysis system. These traces were independently analyzed using the software package used by each submitting agency.

Data Analysis

The sequence traces for one hundred (100) samples were first manually analyzed and compared to the rCRS. For each sample, a consensus report of variants when compared to the rCRS was generated. These reports of variants are available in the U.S. Department of Justice FBI's Laboratory Division “mtDNA Population Database.” The mtDNA sequences of Assessment 2 were analyzed by the automated data analysis system and compared to the “mtDNA Population Database.” The automated data analysis system is not defined by the same base-calling rules as those used for the input of mtDNA profiles into the “mtDNA Population Database”; therefore, more ambiguous base positions are detected with the automated data analysis system.

As described in Example 1, the same set of traces was then processed by the automated data analysis system according to the procedure described above. A side-by-side comparison of the manual analysis and the automated data analysis system results was performed. All differences between manual review consensus and the automated calls were counted. The numbers of true positive (TP), false positive (FP), true negative (TN), and false negative (FN) bases were determined. The sensitivity, specificity, positive predictive value, and negative predictive value of the automated data analysis system were then calculated based on the formulae described in Example 1.

Results

The results, summarized in Table 2, demonstrate that the automated data analysis system provided 99.27% specificity and 99.99% negative predictive value on the test sample sets. The sensitivity evaluation indicated that the ability of the automated system to accurately identify ambiguous positions is reasonable. When comparing the number of false positives versus the number of false negatives, and the positive predictive value, this analysis demonstrates that it would be more likely for the automated data analysis system to call an unambiguous base as ambiguous than vice versa.

The results of the test sample sets demonstrate that the automated data analysis system is not likely to produce mitochondrial profiles that would result in a false exclusion.

Those of skill will further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled persons may implement the described functionality in varying ways for each particular application, but such implementation decisions should not be interpreted as causing a departure from the scope of the present invention.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any processor, controller, microcontroller, or state machine. A processor may also be implemented as a combination of computing devices, for example, a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC.

The above description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

What is claimed is:

1. An automated method for sequence evaluation used to compare sequence information relating to at least one sample against sequence information relating to at least one reference, the method comprising:
acquiring sequence information relating to the at least one sample and to the at least one reference;
evaluating the sequence information relating to the at least one sample to identify ambiguous bases present within the sample sequence information by applying a rule-based criteria wherein ambiguous bases are distinguished from unambiguous bases on the basis of the following criteria: (a) scan position differences, (b) peak height ratios, (c) peak area ratios, and (d) base composition; and
evaluating the quality and coverage of the sample sequence information in comparison to the reference sequence information to identify reportable ranges and sequence variants for the sample sequence information.

2. The method of claim 1, wherein the rule-based criteria for assessing scan position differences to differentiate between ambiguous and unambiguous bases further comprises identifying differences between scan positions of major and minor bases within the sample sequence information which fall below an empirical threshold.

3. The method of claim 2, wherein the empirical threshold associated with identifying differences between scan positions is in the range of approximately 0 to approximately 3.

4. The method of claim 1, wherein the rule-based criteria for assessing scan position differences for differentiating between ambiguous and unambiguous bases further comprises identifying differences between scan positions of major and minor bases within the sample sequence information which reside above, below, or are substantially equivalent to a user-defined threshold.

5. The method of claim 1, wherein the rule-based criteria for assessing peak height ratios to differentiate between ambiguous and unambiguous bases further comprises assessing peak height ratios for major and minor bases within the sample sequence information which exceed an empirical threshold.

6. The method of claim 5, wherein the empirical threshold associated with assessing peak height ratios is in the range of approximately 0.3 to approximately 1.0.

7. The method of claim 1, wherein the rule-based criteria for assessing peak height ratios for differentiating between ambiguous and unambiguous bases further comprises assessing peak area ratios of major and minor bases within the sample sequence information which reside above, below, or are substantially equivalent to a user-defined threshold.

8. The method of claim 1, wherein the rule-based criteria for assessing peak area ratios to differentiate between ambiguous and unambiguous bases further comprises assessing peak area ratios for major and minor bases within the sample sequence information which exceed an empirical threshold.

9. The method of claim 8, wherein the empirical threshold associated with assessing peak area ratios is in the range of approximately 0.3 to approximately 1.0.

10. The method of claim 1, wherein the rule-based criteria for assessing peak area ratios for differentiating between ambiguous and unambiguous bases further comprises assessing peak area ratios of major and minor bases within the sample sequence information which reside above, below, or are substantially equivalent to a user-defined threshold.

11. The method of claim 1, wherein the rule-based criteria for assessing base composition to differentiate between ambiguous and unambiguous bases further comprises determining if the major and minor bases within the sample sequence information are both purines or both pyrimidines.

12. The method of claim 11, wherein when ambiguity in the base composition is increased when the major and minor bases are both purines or both pyrimidines.

13. The method of claim 1, wherein the rule-based criteria for distinguishing between ambiguous and unambiguous bases comprises identifying consecutive runs of bases exceeding an empirical threshold.

14. The method of claim 13, wherein the empirical threshold associated with comprises identifying consecutive runs of bases is in the range of approximately 10 to approximately 13.

15. The method of claim 1, wherein the rule-based criteria for distinguishing between ambiguous and unambiguous bases identifies consecutive runs of bases which reside above, below, or are substantially equivalent to a user-defined threshold.

16. The method of claim 1, wherein identified ambiguous bases are excluded from the evaluation of quality and coverage of the sample sequence information.

17. The method of claim 1, wherein identified ambiguous bases are excluded from the identification of sequence variants.

18. The method of claim 1, wherein the sequence information corresponds to mitochondrial DNA sequence information.

19. An automated method for mitochondrial DNA analysis used to identify associations between a target sample of unknown familial origin with that of at least one reference sample, the method comprising:
acquiring genetic information describing the sequence composition and characteristics for a plurality of nucleotides relating to the mitochondrial genetic makeup of the target sample and at least one reference sample;
assessing the genetic information to identify a degree of ambiguity associated with each of the plurality of nucleotides wherein ambiguous nucleotides are distinguished from unambiguous nucleotides on the basis of: (a) scan position differences, (b) peak height ratios, (c) peak area ratios, and (d) nucleotide compositions;
comparing the genetic information and the degree of ambiguity associated with each of the plurality of nucleotides of the target sample and the at least one reference sample to identify a nucleotide signature that provides distinguishing information used to identify sequence similarities and differences between the target sample and the at least one reference sample; and
comparing the nucleotide signature of the target sample to that of the at least one reference sample such that substantially identical nucleotide signatures identify the target sample as being of the same familial origin as the at least one reference sample and nucleotide signatures which are not substantially identical identify the target sample as not being of the same familial origin as the at least one reference sample.

20. The method of claim 19, wherein the genetic information comprises, in part, raw sequence information selected from the group consisting of: electropherogram/sequence traces, information describing peak characteristics, peak height information, peak area information, peak width information, putative nucleotide identifications, base calls, quality value assessments, and scan positions.

21. The method of claim 19 wherein, nucleotides associated with a threshold degree of ambiguity are excluded from inclusion in the nucleotide signature.

22. The method of claim 19, wherein the genetic information relates to one or more hypervariable regions within the mitochondrial genome.

23. The method of claim 19, wherein the origin of the genetic information for the target sample and/or the reference sample is skin, hair, salvia, semen, tissue, bone, or blood.

24. The method of claim 19, wherein the genetic information for the reference sample comprises sequence information obtained from an original Cambridge reference sequence database or a revised Cambridge Reference Sequence database.

25. The method of claim 19, wherein the use of scan position differences to differentiate between ambiguous and unambiguous nucleotides further comprises identifying differences between scan positions of major and minor bases within the sample sequence information which fall below an empirical threshold.

26. The method of claim 19, wherein the use of scan position differences for differentiating between ambiguous and unambiguous nucleotides further comprises identifying differences between scan positions of major and minor bases within the sample sequence information which reside above, below, or are substantially equivalent to a user-defined threshold.

27. The method of claim 19, wherein the use of peak height ratios to differentiate between ambiguous and unambiguous nucleotides further comprises assessing peak height ratios for major and minor bases within the sample sequence information which exceed an empirical threshold.

28. The method of claim 19, wherein the use of peak height ratios for differentiating between ambiguous and unambiguous nucleotides further comprises assessing peak area ratios of major and minor bases within the sample sequence information which reside above, below, or are substantially equivalent to a user-defined threshold.

29. The method of claim 19, wherein the use of peak area ratios to differentiate between ambiguous and unambiguous nucleotides further comprises assessing peak area ratios for major and minor bases within the sample sequence information which exceed an empirical threshold.

30. The method of claim 19, wherein the use of peak area ratios for differentiating between ambiguous and unambiguous nucleotides further comprises assessing peak area ratios of major and minor bases within the sample sequence information which reside above, below, or are substantially equivalent to a user-defined threshold.

31. The method of claim 19, wherein the use of nucleotide composition to differentiate between ambiguous and unambiguous nucleotides further comprises determining if the major and minor bases within the sample sequence information are both purines or both pyrimidines.

32. The method of claim 31, wherein when ambiguity in the nucleotide composition is increased when the major and minor bases are both purines or both pyrimidines.

33. The method of claim 19, wherein ambiguous and unambiguous nucleotides are distinguished, in part, by identifying consecutive runs of nucleotides exceeding an empirical threshold.

34. The method of claim 19, wherein ambiguous and unambiguous nucleotides are distinguished, in part, by identifying consecutive runs of nucleotides which reside above, below, or are substantially equivalent to a user-defined threshold.

35. A system for conducting automated comparison analyses of sequence information relating to at least one sample and at least one reference, the system comprising:
a setup module that acquires and formats sequence information relating to the at least one sample and the at least one reference;
a trace-analysis module that prepares the sequence information for comparison and includes functionality to select appropriate regions of the sequence information of the at least one sample and the at least one reference for subsequent comparison;
an assembly-analysis module that generates one or more consensus sequences between the at least on sample and the at least one reference and includes functionality for evaluating the sequence information to distinguish between ambiguous and unambiguous nucleotides within the sequence information on the basis of the following criteria: (a) scan position differences, (b) peak height ratios, (c) peak area ratios, and (d) nucleotide composition; and
a variant-analysis module that generates a nucleotide profile which details the results of comparing the at least one sample and the at least one reference and identifies nucleotide variations between the at least one sample and the at least one reference wherein the variants may be used to determine the degree of similarity between the at least one sample and the at least one reference.

36. The system of claim 35, wherein the use of scan position differences by the assembly-analysis module to differentiate between ambiguous and unambiguous nucleotides is conducted by identifying differences between scan positions of major and minor bases within the sample sequence information which fall below an empirical threshold.

37. The system of claim 35, wherein the use of scan position differences by the assembly-analysis module to differentiate between ambiguous and unambiguous nucleotides further comprises identifying differences between scan positions of major and minor bases within the sample sequence information which reside above, below, or are substantially equivalent to a user-defined threshold.

38. The system of claim 35, wherein the use of peak height ratios by the assembly-analysis module to differentiate between ambiguous and unambiguous nucleotides further comprises assessing peak height ratios for major and minor bases within the sample sequence information which exceed an empirical threshold.

39. The system of claim 35, wherein the use of peak height ratios by the assembly-analysis module to differentiate between ambiguous and unambiguous nucleotides further comprises assessing peak area ratios of major and minor bases within the sample sequence information which reside above, below, or are substantially equivalent to a user-defined threshold.

40. The system of claim 35, wherein the use of peak area ratios by the assembly-analysis module to differentiate between ambiguous and unambiguous nucleotides further comprises assessing peak area ratios for major and minor bases within the sample sequence information which exceed an empirical threshold.

41. The system of claim 35, wherein the use of peak area ratios by the assembly-analysis module to differentiate between ambiguous and unambiguous nucleotides further comprises assessing peak area ratios of major and minor bases within the sample sequence information which reside above, below, or are substantially equivalent to a user-defined threshold.

42. The system of claim 35, wherein the use of nucleotide composition by the assembly-analysis module to differentiate between ambiguous and unambiguous nucleotides further comprises determining if the major and minor bases within the sample sequence information are both purines or both pyrimidines.

43. A computer readable medium having stored thereon instructions which cause a general purpose computer to perform the steps of:
acquiring sequence information relating to at least one sample and to at least one reference for purposes of comparison;
evaluating the sequence information relating to the at least one sample to identify ambiguous bases present within the sample on the basis of the following criteria: (a) scan position differences, (b) peak height ratios, (c) peak area ratios, and (d) base composition; and
evaluating the quality and coverage of the sample sequence information in comparison to the reference sequence information to identify reportable ranges and sequence variants for the sample sequence information.

44. A computer-based system for performing automated sequence evaluation and used to identify associations between a target sample of unknown familial origin with that of at least one reference sample, the system comprising:
a database for storing genetic information describing the sequence composition and characteristics for a plurality of nucleotides relating to the mitochondrial genetic makeup of the target sample and at least one reference sample;
a program which performs the operations of:
assessing the genetic information to identify a degree of ambiguity associated with each of the plurality of nucleotides wherein ambiguous nucleotides are distinguished from unambiguous nucleotides on the basis of: (a) scan position differences, (b) peak height ratios, (c) peak area ratios, and (d) nucleotide compositions;
comparing the genetic information and the degree of ambiguity associated with each of the plurality of nucleotides of the target sample and the at least one reference sample to identify a nucleotide signature that provides distinguishing information used to identify sequence similarities and differences between the target sample and the at least one reference sample; and
comparing the nucleotide signature of the target sample to that of the at least one reference sample such that substantially identical nucleotide signatures identify the target sample as being of the same familial origin as the at least one reference sample and nucleotide signatures which are not substantially identical identify the target sample as not being of the same familial origin as the at least one reference sample.
